(19) United States
(12) Patent Application Publication
(10) Pub. No.: US 2007/0122341 A1
(43) Pub. Date: May 31, 2007

(54) Benzylideneaniline derivatives and their radioisotope labeled compounds for binding and imaging of beta-amyloid plaques

(75) Inventors: Jae Min Jeong, Seoul 136-060 (KR); Dong Soo Lee, Seoul 137-868 (KR); June-Key Chung, Seoul 138-902 (KR); Myung Chul Lee, Seoul 138-160 (KR)

Correspondence Address: HESLIN ROTHENBERG FARLEY & MESITI PC, 5 COLUMBIA CIRCLE, ALBANY, NY, 12203, US

(73) Assignee: SEOUL NATIONAL UNIVERSITY INDUSTRY FOUNDATION, Seoul (KR)

(21) Appl. No.: 11/424,741

(22) Filed: Jun. 16, 2006

(30) Foreign Application Priority Data
Nov. 29, 2005 (KR) 10-2005-0115012

(51) Int. Cl. A61K 51/00; C07D 249/02

(52) U.S. Cl. 424/1.11; 564/272

(57) ABSTRACT

Benzylideneaniline derivatives of formula 1

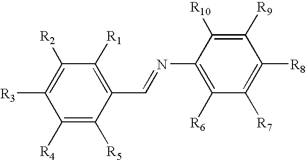

wherein R1-R5 are independently selected from hydrogen, C₁-C₄ alkyl and F (at least one of them is F) and each R₆-R₁₀ are independently selected from hydrogen, C₁-C₄ alkyl, OH, OCH₃, NH₂, NHCH₃ and N(CH₃)₂ (at least one of them is OH, OCH₃, NH₂, NHCH₃ or N(CH₃)₂) are disclosed. Benzylideneaniline derivatives according to the present invention have high affinity to β-amyloid plaques. Thus, they can cross the blood-brain-barrier (BBB) and bind to β-amyloid plaques after administration into the body, making them useful for treatment, prevention, or imaging of Alzheimer's disease.

### CROSS-REFERENCE TO RELATED APPLICATIONS

This application claims the priority of Korean Patent Application No.: 10-2005-0115012 filed Nov. 29, 2005. The contents of the priority application are hereby incorporated by reference in their entirety.

### TECHNICAL FIELD

The present invention relates to novel benzylideneaniline derivatives for β-amyloid plaque imaging, their radioisotope labeled compounds and their preparation method.

### BACKGROUND OF THE INVENTION

Alzheimer's disease is characterized by a decrease of brain nerve cells resulting in reduced memory and cognitive power. Plaques or tangles that are formed by aggregation of β-amyloid peptide are found in the Alzheimer's patients' brain.

Alzheimer's disease might be suppressed by administration of drugs inhibiting formation of β-amyloid plaques and tangles.

Although, Alzheimer's disease can be confirmed by staining the postmortem brain with Congo red, it cannot be applied to a live human. Congo red cannot enter into brain when it is administrated to the human body, because it is impermeable to the blood-brain-barrier (BBB) due to high hydrophilicity. Thus, in order to image and diagnose Alzheimer's disease it is necessary to radiolabel a BBB-permeable compound that can bind to β-amyloid plaques.

The earliest radiolabeled compounds for imaging β-amyloid plaques were Chrysamine-G derivatives as shown in Formula 2 and 3. However, these compounds were not practically applicable due to low BBB-permeability. (Klunk W E, Debnath M L, Pettegrew J W. Development of small molecule probes for the beta-amyloid protein of Alzheimer's disease. Neurobiol Aging 1994; 15:691-8. Klunk W E, Debnath M L, Pettegrew J W. Chrysamine-G binding to Alzheimer and control brain: Autopsy study of a new amyloid probe. Neurobiol Aging 1995; 16:541-8.)

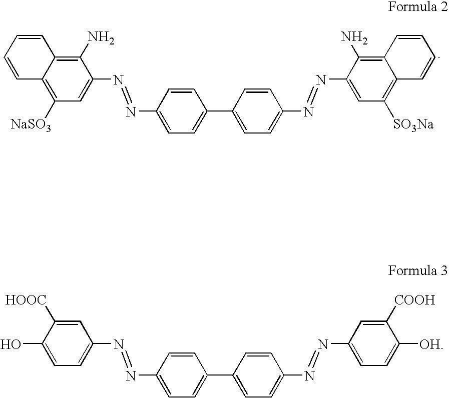

The research became more active after the development of 6-dialkylamino-2-naphthylethylidene (FDDNP) of Formula 5 and thioflavin-T derivatives of Formula 6. (Agdeppa E D, Kepe V, Liu J, Flores-Torres S, Satyamurthy N, Petric A, Cole G M, Small G W, Huang S C, Barrio J R. Binding characteristics of radiofluorinated 6-dialkylamino-2-naphthylethylidene derivatives as positron emission tomography imaging probes for β-amyloid plaques in Alzheimer's disease. J Neuroscience 2001; 21:1-5. Mathis C A, Bacskai B J, Kajdasz S T, MlLellan M E, Frosch M P, Hyman B T, Holt D P, Wang Y, Huang G-F, Debnath M L, Klunk W E. A lipophilic thioflavin-T derivative for positron emission tomography (PET) imaging of amyloid in brain. Bioorg Med Chem Lett 2002; 12:295-298.)

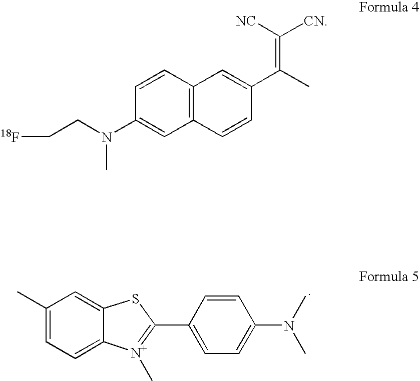

Benzothiazole derivative of Formula 6 and stilbene derivatives of Formula 7 were patented as radiolabeled agents for β-amyloid plaque imaging. (US Patent Pub. No. 2002/0133019 A1, W. E. Klunk, C. A. Mathis Jr, Thioflavin derivatives for use in antemortem diagnosis of Alzheimer's disease and vivo imaging and prevention of amyloid deposition; US Patent Pub. No. 2003/0149250 A1, H. F. Kung, M-P. Kung, Z-P. Zhuang, Stilbene derivatives and their use for binding and imaging amyloid plaques).

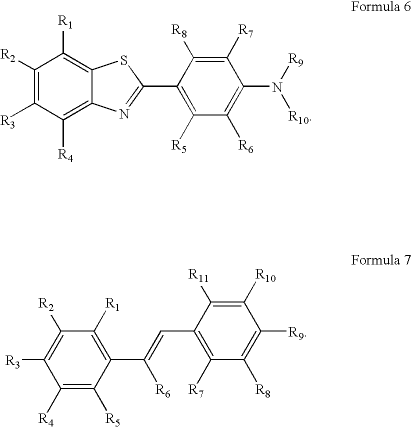

The present invention includes benzylideneaniline as a basic chemical structure that can easily penetrate BBB due to small molecular size and high lipophilicity. In addition the compounds can be used for diagnosis and treatment of Alzheimer's disease due to high affinity to β-amyloid plaques.

All the positron-emitting agents for imaging β-amyloid plaques developed until now have shortcomings. In case of ¹¹C labeled compounds, the short half-life of ¹¹C (20 min) is a limiting factor. Because, it would seriously decay for imaging after 1 hr waiting time that is required for enough uptake for imaging in the brain. In addition, commercialization also would be difficult due to time-consuming transportation. The reported compounds labeled with ¹⁸F that has a relatively long half-life (110 min) also have problems in practical use due to release of 18F from aliphatic side chain after metabolism.

### TECHNICAL PROBLEM

The technical object of the present invention is to develop compounds that have enough high lipophilicity for blood-brain-barrier (BBB) penetration and enough high affinity to β-amyloid plaques.

Another technical object is to develop practically applicable radiolabeled compounds without the problems of the prior art compounds. In addition, the present invention provides diagnostic method of Alzheimer's disease using the above radiolabeled compounds.

### DISCLOSURE OF THE INVENTION

The present invention comprises the compounds of benzylideneaniline derivatives for β-amyloid plaque imaging, their radiolabeled compounds and their preparation methods.

The first embodiment of the present invention is the benzylideneaniline derivatives described as Formula 1 for imaging β-amyloid plaques:

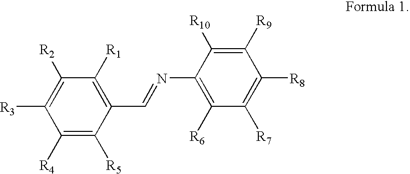

wherein R₁-R₅ are independently selected from hydrogen, C₁-C₄ alkyl and F (at least on of them is F) and each R₆-R₁₀ are independently selected from hydrogen, C₁-C₄ alkyl, OH, OCH₃, NH₂, NHCH₃ and N(CH₃)₂ (at least one of them is OH, OCH₃, NH₂, NHCH₃ or N(CH₃)₂).

Benzylideneaniline derivatives according to the present invention have high affinity to β-amyloid plaques. Thus as they can pass blood-brain-barrier (BBB) and bind to β-amyloid plaques after administration into the body, so they can be used for treatment, prevention, or imaging of Alzheimer's disease.

To image β-amyloid plaques in the alive Alzheimer's patients' brain, administration of a compound radiolabeled with an adequate radioisotope would be the most preferred method. The first choice for this purpose is ¹⁸F, which emits positron with 110 min half-life. 18F-labeled agents would show excellent image in positron emission tomography (PET).

The inventors of the present invention have developed successfully the novel compounds having high in vivo stability and relatively long half-life compared to ¹¹C, by labeling with ¹⁸F at the side chain of aromatic ring.

In benzylideneaniline derivatives according to the present invention, one of R₁-R₅ can be ¹⁸F. Preferably, R₁-R₅ which are not ¹⁸F are all hydrogen, and more preferably, each R₆-R₁₀ are independently selected from hydrogen, OH, OCH₃, NH₂, NHCH₃ and N(CH₃)₂, wherein at least one of them is selected from OH, OCH₃, NH₂, NHCH₃ and N(CH₃)₂.

Brain image of Alzheimer's patient having β-amyloid plaques in the brain can be obtained by PET using one of radiolabeled compounds selected from described above.

In this case, the dissociation constant (K_{D}) of benzylideneaniline compound of the present invention to β-amyloid plaques preferably is 0.00001-10 μM.

The second embodiment of the present invention is about pharmaceutical composition for injection comprising a benzylideneaniline derivative or its radioisotope labeled compound according to the present invention. Radioactivity of the above radiolabeled compound preferably is 0.1-100 mCi at the moment of administration.

The third embodiment of the present invention is about pharmaceutical composition for imaging β-amyloid plaques, comprising a benzylideneaniline derivative or its radioisotope labeled compound according to the present invention.

The forth embodiment of the present invention is about composition for diagnosis of Alzheimer's disease comprising a benzylideneaniline derivative or its radioisotope labeled compound according to the present invention.

The fifth embodiment of the present invention is about method for diagnosis of Alzheimer's disease comprising the use of a benzylideneaniline derivative or its radioisotope labeled compound according to the present invention.

The present invention relates not only to ¹⁸F-labeled benzylideneaniline derivatives but also to labeling method.

According to the present invention, an example of ¹⁸F-labeling method is described in Scheme 1: wherein ¹⁸F is labeled to fluorobenzaldehyde at first and then it is conjugated with an adequate aniline derivative by formation of Schiff's base. Thus labeled compound can be administered to human body after purification by high performance liquid chromatography (HPLC).

According to the present invention, a labeling method of benzylideneaniline derivative with ¹⁸F at R₃ position is described as an example. ¹⁸F-labeled fluorobenzaldehyde of Formula 8 is conjugated with an amine of Formula 9 resulting in an ¹⁸F-labeled benzylideneaniline derivative of Formula 10. ¹⁸F-labeled fluorobenzaldehyde of Formula 8 can be prepared by reacting trimethylammonium benzaldehyde of Formula 11 with ¹⁸F-labeled tetrabutylammonium fluoride in dimethylsulfoxide (DMSO).

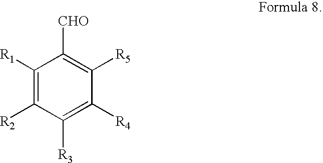

wherein R₁-R₅ are independently selected from hydrogen, C₁-C₄ alkyl and F (at least one of them is ¹⁸F).

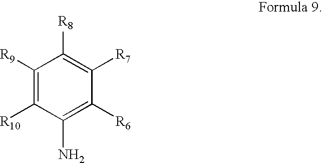

wherein R₆-R₁₀ are independently selected from hydrogen, C₁-C₄ alkyl, OH, OCH₃, NH₂, NHCH₃ and N(CH₃)₂ (at least one of them is OH, OCH₃, NH₂, NHCH₃ or N(CH₃)₂).

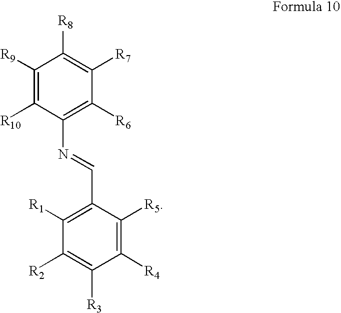

wherein R₁-R₅ are independently selected from hydrogen, C₁-C₄ alkyl and F (at least one of them is ¹⁸F), and R₆-R₁₀ are independently selected from hydrogen, C₁-C₄ alkyl, OH, OCH₃, NH₂, NHCH₃ and N(CH₃)₂ (at least one of them is OH, OCH₃, NH₂, NHCH₃ or N(CH₃)₂).

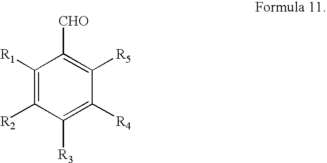

wherein R₁-R₅ are independently selected from hydrogen, C₁-C₄ alkyl and —N(CH₃)₃⁺⁻OTf (at least one of them is —N(CH₃)₃⁺⁻OTf).

According to the present invention, a specific example of synthesizing one of the ¹⁸F-labeled benzylideneaniline derivatives is presented in Scheme 1.

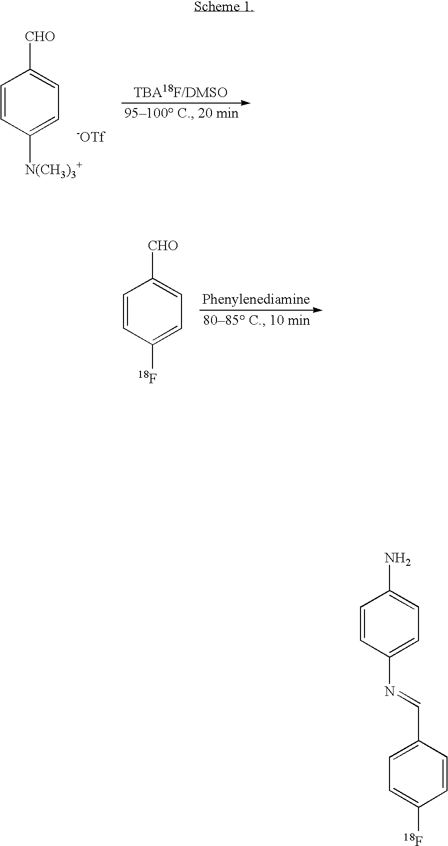

Scheme 1 shows that ¹⁸F-labeled fluorobenzaldehyde is synthesized by reacting trimethylammoniumbenzaldehyde and ¹⁸F-labeled tetrabutylammonium fluoride in DMSO and then it is conjugated with phenylenediamine to produce the final product ¹⁸F-labeled fluorobenzylideneaniline derivative.

In the present invention, benzylideneaniline derivatives labeled by the above method was found to be excellent for PET imaging β-amyloid plaque deposited brain.

The present invention provides lipophilic benzylideneaniline derivatives that have therapeutic or prevention effect for Alzheimer's disease by dissociating or blocking formation of β-amyloid plaques. Fluorescence image of β-amyloid plaques formed in the Alzheimer's patients' brains can be obtained using the fluorescence of benzylideneaniline derivatives. In addition, it is preferred to use the compounds labeled with positron emitter for imaging β-amyloid plaques formed in the brain.

### BRIEF EXPLANATION OF DRAWINGS

FIG. 1. Biodistribution results of the compounds A-F in Example 7.

FIG. 2. Fluorescent imaging of Tg 2576 mouse brain using compound A in Example 7.

FIG. 3. Fluorescent imaging of Tg 2576 mouse brain using compound B in Example 7.

FIG. 4. Fluorescent imaging of Tg 2576 mouse brain using compound C in Example 7.

FIG. 5. Fluorescent imaging of Tg 2576 mouse brain using compound D in Example 7.

FIG. 6. Fluorescent imaging of Tg 2576 mouse brain using compound E in Example 7.

FIG. 7. Fluorescent imaging of Tg 2576 mouse brain using compound F in Example 7.

### BEST MODE FOR CARRYING OUT THE INVENTION

The following examples are given to illustrate the present invention. It should be understood, however, that the invention is not to be limited to the specific conditions or details described in these examples.

### Example 1

### Synthesis of N-(4-fluorobenzylidene)benzene-1,4-diamine

0.01 mol of p-fluorobenzaldehyde and 0.01 mol of phenylenediamine were dissolved in 15 mL of ethanol and refluxed for 6 hours. After cooling of the reaction mixture, the yellowish precipitate was collected and recrystallized in ethanol. Yield: 53%; mp 169-170□; ¹H NMR (CDCl₃) δ 8.47 (s, 1H, N═CH), 7.923 (d, 2H, H_{3′-5′}J=8.7 Hz), 7.906 (d, 2H, H_{3″-5″}J=8.4 Hz), 7.13-7.268 (m, 4H, H_{3′-5′}& H_{3″-5″}); ¹³C NMR (CDCl₃): δ 158.18, 149.78, 130.81, 130.68, 121.81, 116.09, 115.81; LC/MS (m/z) 215 [M+H].

### Example 2

### Synthesis of N-(4-fluorobenzylidene)-N′-methylbenzene-1,4-diamine

0.01 mol of p-fluorobenzaldehyde and 0.01 mol of N-methylphenylenediamine were dissolved in 15 mL of ethanol and refluxed for 6 hours. After removal of the solvent, the yellowish precipitate was purified by collected preparative TLC (ethyl acetate/n-hexane 1/9) and successive crystallization. Yield 17%; mp 106-107□; ¹H NMR (CDCl₃) δ 8.45 (s, 1H, N═CH), 6.61-7.88 (m, 8H, Ar—H), 3.8 (br, s, NH), 2.86 (s, 3H, CH₃); ¹³C NMR (CDCl₃): δ 154.38, 148.78, 141.41, 133.37, 130.208, 122.38, 115.88, 115.59, 112.69, 30.85; LC/MS (m/z) 229 [M+H].

### Example 3

### Synthesis of N-(4-fluorobenzylidene)-N′,N′-dimethylbenzene-1,4-diamine

0.01 mol of p-fluorobenzaldehyde and 0.01 mol of N,N-dimethylphenylenediamine were dissolved in 15 mL of ethanol and refluxed for 6 hours. After cooling of the reaction mixture, the yellowish precipitate was collected and recrystallized in ethanol. Yield 9.1%; mp 191-192□; ¹H NMR (CDCl₃) δ 8.47 (s, 1H, N═CH), 6.73-7.8 (m, 8H, Ar—H), 2.98 (s, 6H, CH₃); ¹³C NMR (CDCl₃) δ 154.36, 148.34, 130.2, 130.09, 122.21, 115.89, 115.596, 112.86, 40.72; LC/MS (m/z) 243 [M+H].

### Example 4

### Synthesis of 4-(4-fluorobenzylidene)amino]phenol

0.01 mol of p-fluorobenzaldehyde and 0.01 mol of p-aminophenol were dissolved in 15 mL of ethanol and refluxed for 6 hours. After cooling of the reaction mixture, the gray precipitate was collected and recrystallized in benzene and ethyl acetate mixture. Yield 55%; mp 148-149□(Lit. 145-146□); ¹H NMR (CDCl₃) (DMSO-d₆) δ 9.5 (br s, OH), 8.58 (s, 1H, N═CH), 7.27-7.95 (m, 4H, H_{2′-6′}& H_{2″-6″}) 7.181 (d, 2H, H_{3′-5′} J=9 Hz), 6.791 (d, 2H, H_{3″-5″} J=8.4 Hz); ¹³C NMR (DMSO-d₆) δ 165.35, 162.06, 156.36, 142.5, 133.2, 130.6, 122.56, 115.78, 115.72; LC/MS (m/z) 216 [M+H].

### Example 5

### Synthesis of (4-fluorobenzylidene)-(4-methoxyphenyl)amine

0.01 mol of p-fluorobenzaldehyde and 0.01 mol of p-methoxyaniline were dissolved in 15 mL of ethanol and refluxed for 6 hours. After cooling of the reaction mixture, the precipitate was collected and recrystallized in benzene and ethyl acetate mixture. Yield 38.9%; mp 92-93□ (Lit. 93-95□); ¹H NMR (CDCl₃) (DMSO-d₆) δ 8.607 (s, 1H, N═CH), 7.287-7.99 (m, 4H, H_{2′-6′} & H_{2″-6″}) 7.273 (d, 2H, H_{3′-5′} J=8.4 Hz), 6.961 (d, 2H, H_{3″-5″} J=9 Hz), 3.57 (s, 3H, OCH₃); ¹³C NMR (DMSO-d₆) δ 158.03, 157.108, 143.971, 133.971, 130.76, 122.468, 116.04, 115.74, 114.47, 55.349; LC/MS (m/z) 230 [M+H].

### Example 6

### Synthesis of 5-[(4-fluorobenzylidene)amino]-2-hydroxybenzoic acid

0.01 mol of p-fluorobenzaldehyde and 0.01 mol of 5-aminosalicylic acid were dissolved in 15 mL of ethanol and refluxed for 6 hours. After cooling of the reaction mixture, 5-aminosalicylic acid remained was removed by filtration. The filtrate was evaporated, and then the residue was collected and recrystallized in n-hexane and ethyl acetate mixture, and purified by preparative TLC (ethyl acetate/n-hexane 1/9). Yield 9%; mp 193□ (decompose); ¹H NMR (DMSO-d₆) δ 9.96 (s, 1H, OH), 8.65 (s, 1H, N═CH), 7.29-7.99 (m, 5H, H_{5′}, H_{2′-6′} & H_{2″-6″}) 6.969 (d, 2H, H_{3″-5″} J=8.7 Hz); ¹³C NMR (DMSO-d₆) δ 171.58, 159.78, 157.603, 142.059, 132.25, 130.75, 130.63, 128.59, 122.098, 117.7, 115.93, 113.88; LC/MS (m/z) 215 [M-45].

### Example 7

### ¹⁸F-Labeling of Benzylideneaniline Derivatives

Method A: The ¹⁸F was eluted from a light QMA SepPak cartridge with 1 mL of 2.3% tetrabutylammonium bicarbonate (TBAB) in 83.8% MeCN and evaporated with 1 mL of MeCN under argon bubbling at 95-100° C. After completion of the evaporation, 5 mg of triflate salt of 4-(N,N,N-trimethylamino)benzaldehyde in 1 mL of dry dimethylsulfoxide (DMSO) was added and heated at 90-100° C. for 15 min for ¹⁸F-labeling. The reaction mixture was poured into 15 mL of water with mixing. The solution was successively passed through two ICH cartridges, a QMA SepPak cartridge and a light C₁₈ SepPak cartridge. 4-[¹⁸F]Fluorobenzaldehyde trapped in C₁₈ SepPak cartridge was eluted with 0.5 mL of EtOH and added to substituted aniline (5 mg: phenylene diamine, methylphenyl diamine, N,N-dimethylphenylene diamine, p-aminophenol, or p-methoxyaniline) in 0.5 mL EtOH and heated at 80-85° C. for 10 min. The reaction mixture was diluted with 1 mL of water and injected to preparative HPLC (eluted with 5% ethanol, 4 mL/min). ¹⁸F-Labeled benzylidineanilines were collected between 13-16 min and analyzed with analytical HPLC.

Method B: The ¹⁸F was eluted from a light QMA SepPak cartridge with 1 mL of 2.3% tetrabutylammonium bicarbonate (TBAB) in 83.8% MeCN and evaporated with 1 mL of MeCN under argon bubbling at 95-100° C. After completion of the evaporation, 5 mg of triflate salt of 4-(N,N,N-trimethylamino)benzaldehyde in 1 mL of dry dimethylsulfoxide (DMSO) was added and heated at 90-100° C. for 15 min, thereby 4-[¹⁸F]fluorobenzaldehyde was synthesized. Then 5 mg of substituted aniline (phenylene diamine, methylphenyl diamine, N,N-dimethylphenylene diamine, p-aminophenol, or p-methoxyaniline) was added and heated for further 20 min. The reaction mixture was diluted with 15 mL of water and successively passed through two ICH cartridges, a QMA SepPak cartridge, and a light C₁₈ SepPak cartridge, and washed with 15 mL of distilled water. The light C₁₈ SepPak cartridge was eluted with 1 mL of EtOH to collect ¹⁸F-labeled benzylideneanilines and purified in preparative HPLC as described above.

The results of ¹⁸F-labeling of benzylideneaniline derivatives and purification were summarized in Table 1.

TABLE 1

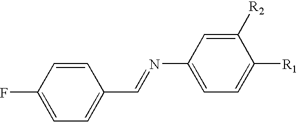

| | | | | Radiochemical yield | |
|---|---|---|---|---|---|
| Compounds | R₁ | R₂ | t_{R} | Method A | Method B |
| A | NH₂ | H | 11.64 | 44.2 | 48.3 |
| B | NHMe | H | 11.66 | 43.2 | 54.2 |
| C | NMe₂ | H | 12.75 | 32.7 | 51.4 |
| D | OH | H | 12.01 | 39.5 | 27.7 |
| E | OMe | H | 14.94 | 44.9 | 45.2 |
| F | OH | COOH | 11.9 | 42.9 | 49.0 |

In Table 1, the radiochemical yield of benzylideneaniline derivatives was 32.7%-44.9% by method A and 27.7%-54.2% by method B, respectively. Except compound D, method B showed better radiochemical yield for ¹⁸F labeling of benzylideneaniline derivatives.

<Experiment 1> In Vitro Binding Assay

Aggregated peptides were prepared using the solid form of peptides Aβ₁₋₄₀ (purchased from Sigma) by a literature method (Klunk, W. E.; Wang, Y.; Huang, G-F.; Debnath, M. L.; Holt D. P.; Mathis, C. A. Uncharged thioflavin-T derivatives bind to amyloid-beta protein with high affinity and readily enter the brain. Life Sci. 2001, 69(13), 1471-1914). The aggregated Aβ₁₋₄₀ peptide was aliquoted and stored at −70° C. Binding studies were performed according to the procedure described in the literature with some modifications (Zhuang, Z.-P.; Kung, M.-P.; Wilson, A.; Lee, C.-W.; Plossl, K.; Hou, C.; Holtzman, D. M.; Kung, H. F.; Structure-activity relationship of imidazo[1,2-a]pyridines as ligands for detecting β-amyloid plaques in the brain. J. Med. Chem. 2003; 46(2), 237-243). The Ki values of benzylideneaniline derivatives were evaluated with Aβ₁₋₄₀ aggregates.

The reaction mixtures containing 100 μL of Aβ¹⁻⁴⁰ aggregates (20 nM in the final mixture), 100 μL of benzylideneaniline derivatives (10⁻⁵-10⁻¹⁰ M in 50% ethanol), 100 μL of [¹²⁵I]3′-I-BTA-1 in 50% ethanol (0.04 nM in the final mixture), and 700 μL of phosphate buffered saline (pH=7.2) were incubated for 3 hr at room temperature, filtered through Whatman GF/B glass filters and washed twice with 3 mL of 10% EtOH. The filters were counted in a gamma-counter. Nonspecific binding was determined by reacting in the presence of 10 μM stilbene. From this inhibition experiment, Ki values of unlabeled compounds were calculated from IC₅₀ values, and summarized in Table 2.

TABLE 2

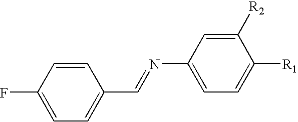

| Compounds | R₁ | R₂ | Kᵢ (nM) |
|---|---|---|---|
| A | NH₂ | H | 304 |
| B | NHMe | H | 1041 |
| C | NMe₂ | H | 149 |
| D | OH | H | >10000 |
| E | OMe | H | >10000 |
| F | OH | COOH | >10000 |

In Table 2, Kᵢ values of compound A, B and C, which have amino group, were 304, 1,041, 149 nM, respectively. This result proved that compound A, B and C have affinity to Aβ₁₋₄₀. Kᵢ values of other compounds were over 10,000 nM, and these compounds have no affinity to Aβ₁₋₄₀.

Experiment 2> Biodistribution in Normal Mice.

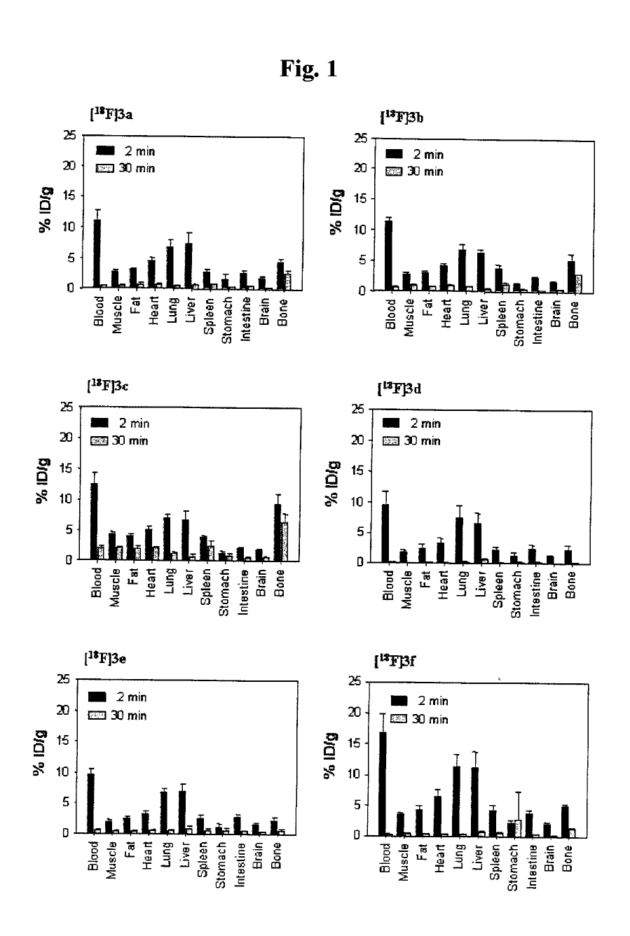

Biodistribution study of the ¹⁸F-labeled compounds of Example 7 was performed with ICR male mice (weight range 27-28 g). 0.1 mL of a saline solution containing each ¹⁸F-labeled benzylideneaniline compound A, B, C, D, E or F (37-74 kBq) was injected through the tail vein. The mice were sacrificed by decapitation at 2 min and 30 min time points post injection. The organs of interest, including blood, muscle, fat, heart, lung, liver, spleen, stomach, intestine, brain and bone, were separated and weighed, and the remaining radioactivity was counted with a NaI well counter. The percentage of injected dose per gram of tissue (% ID/g) was calculated from the data, and summarized in FIG. 1. And the ratios of brain uptake at 2 min over 30 min were summarized in Table 3.

TABLE 3

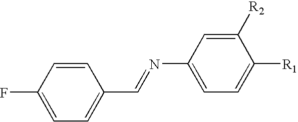

| Compounds | R₁ | R₂ | Brain uptake ratio |
|---|---|---|---|
| A | NH₂ | H | 8.7 ± 1.7 |
| B | NHMe | H | 5.4 ± 0.5 |
| C | NMe₂ | H | 2.9 ± 0.5 |
| D | OH | H | 24.3 ± 4.6 |
| E | OMe | H | 4.2 ± 0.9 |
| F | OH | COOH | 11.0 ± 2.0 |

In Table 3, the compound D showed highest brain uptake ratio, 24.3. However, compound D has low binging affinity to Aβ₁₋₄₀. In the results of in vitro and in vivo experiments, compound A was ideal for imaging of Aβ₁₋₄₀.

<Experiment 3> Fluorescent Staining of Brain Sections of Transgenic Tg 2576 Mouse.

The frozen brain of a 24-month old Tg2576 mouse (male, 30 g) was equilibrated to −20° C. The 20 μm thickness brain sections were obtained using a cryostat microtome and mounted onto silane-coated glass slides, and stored at −70° C. until use. Compound A, B, C, D, E or F was used for fluorescent staining of brain section. The solution (0.0125% in 40% EtOH/60% PBS) of each compound (0.3 mL) was dropped on the brain section, and the section was incubated for 3 min. The slide glass was washed with 50% of ethanol and phosphate buffered saline (PBS) mixture for 3 min, PBS for 1 min and water for 5 min, successively. After drying, the brain section was investigated by fluorescent microscope (excitation filter 350-390 nm, emission filter 530±15 nm).

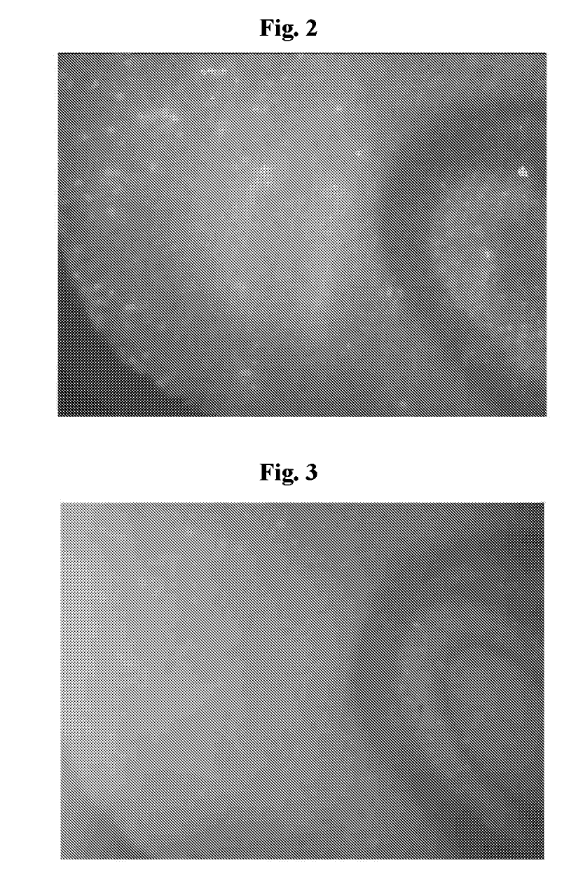

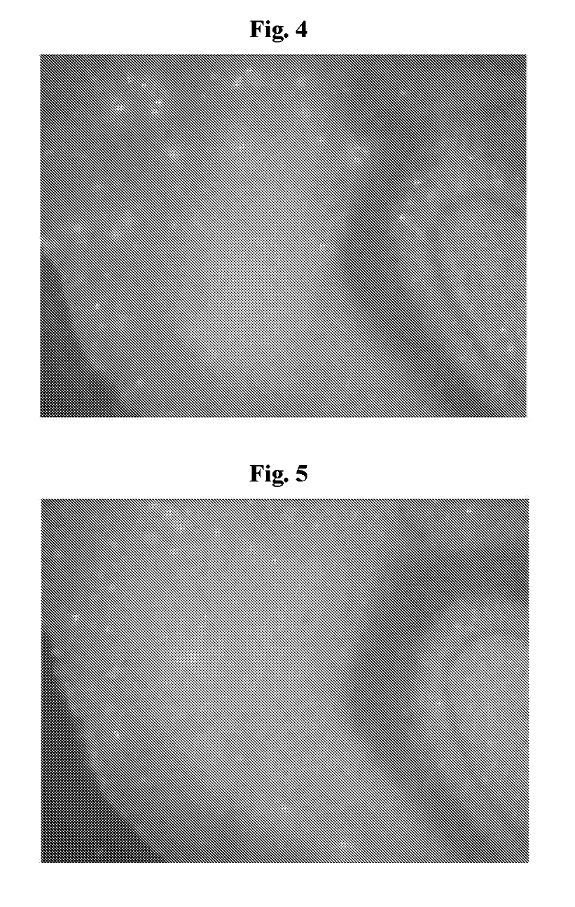

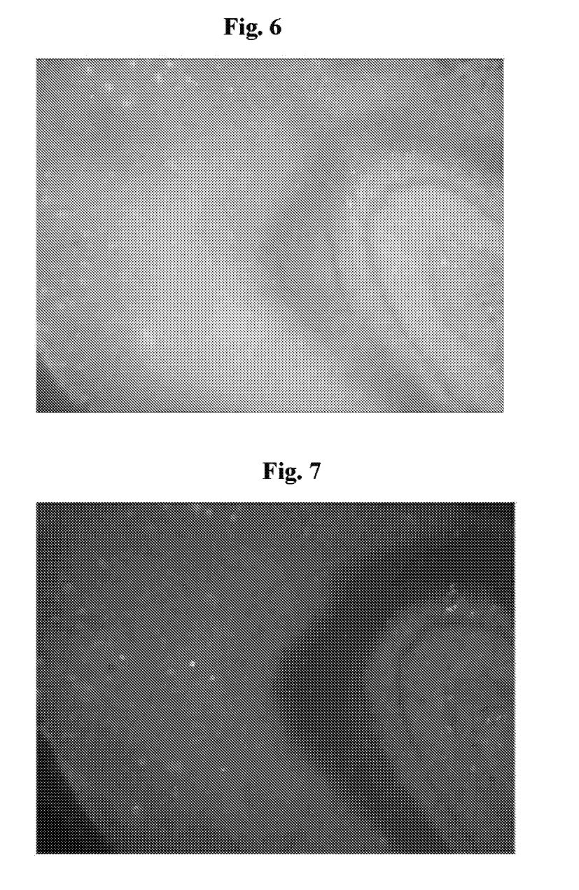

FIG. 2-FIG. 7 showed fluorescent stained brain section images of compound A, B, C, D, E or F.

The bright spots on the images were amyloid plaques, which were combined with benzylideneaniline derivatives.

### EFFECT OF INVENTION

As described above, benzylideneaniline derivatives of the present invention can be easily labeled with radioisotopes. And the benzylideneaniline derivatives and their radiolabeled compounds of the present invention have excellent features for β-amyloid plaque imaging such as high affinity to β-amyloid plaques, high initial brain uptake and rapid clearance from the brain due to high BBB permeability.

The compounds of the present invention can be used for imaging β-amyloid plaques by binding to the deposited β-amyloid plaques in the brain of Alzheimer's disease patients. And these compounds also can be used for diagnosis, prevention or therapy of Alzheimer's disease caused by of β-amyloid plaques.

1. A benzylideneaniline derivative described as Formula 1: 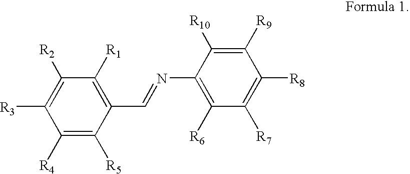
wherein R₁-R₅ are independently selected from hydrogen, C₁-C₄ alkyl and F (at least on of them is F), and each R₆-R₁₀ are independently selected from hydrogen, C₁-C₄ alkyl, OH, OCH₃, NH₂, NHCH₃ and N(CH₃)₂ (at least one of them is OH, OCH₃, NH₂, NHCH₃ or N(CH₃)₂).

2. The benzylideneaniline derivative according to claim 1, wherein one of R₁-R₅ is ¹⁸F.

3. The benzylideneaniline derivative according to claim 2, wherein R₁-R₅ that are not ¹⁸F are all hydrogen.

4. The benzylideneaniline derivative according to claim 3, wherein R₆-R₁₀ are independently selected from hydrogen, OH, OCH₃, NH₂, NHCH₃ and N(CH₃)₂, wherein at least one of them is selected from OH, OCH₃, NH₂, NHCH₃ and N(CH₃)₂.

5. The benzylideneaniline derivative according to claim 1, wherein the dissociation constant (K_{D}) of benzylideneaniline compound to β-amyloid plaques preferably is 0.00001-10 μM.

6. A pharmaceutical composition for injection, which comprises a benzylideneaniline derivative of claim 1.

7. A pharmaceutical composition for injection, which comprises a benzylideneaniline derivative of claim 2 as the amount of radioactivity of 0.1-100 mCi at the moment of administration.

8. A pharmaceutical composition for imaging β-amyloid plaques, which comprises a benzylideneaniline derivative of claim 1.

9. A composition for diagnosis of Alzheimer's disease, which comprises a benzylideneaniline derivative of claim 1.

10. A method for diagnosis of Alzheimer's disease, which comprises the use of a benzylideneaniline derivative of claim 1.

11. A preparation method of ¹⁸F-labeled benzylideneaniline derivative of Formula 10, which comprises a step of conjugating ¹⁸F-labeled fluorobenzaldehyde of Formula 8 with an amine of Formula 9: 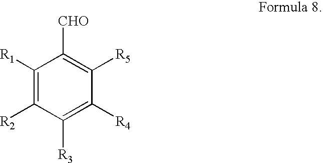
[wherein R₁-R₅ are independently selected from hydrogen, C₁-C₄ alkyl and F (at least one of them is ¹⁸F)] 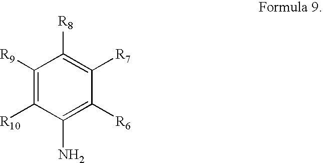
[wherein R₆-R₁₀ are independently selected from hydrogen, C₁-C₄ alkyl, OH, OCH₃, NH₂, NHCH₃ and N(CH₃)₂ (at least one of them is OH, OCH₃, NH₂, NHCH₃ or N(CH₃)₂)] 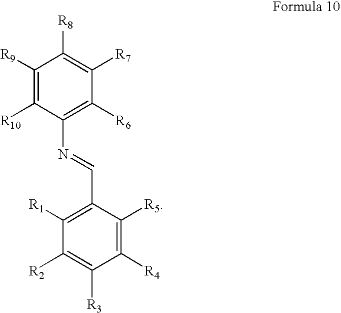
[wherein R₁-R₅ are independently selected from hydrogen, C₁-C₄ alkyl and F (at least one of them is ¹⁸F), and R₆-R₁₀ are independently selected from hydrogen, C₁-C₄ alkyl, OH, OCH₃, NH₂, NHCH₃ and N(CH₃)₂ (at least one of them is OH, OCH₃, NH₂, NHCH₃ or N(CH₃)₂)]

12. The preparation method according to claim 11, wherein the ¹⁸F-labeled fluorobenzaldehyde of Formula 8 is prepared by reacting trimethylammonium benzaldehyde of Formula 11 with ¹⁸F-labeled tetrabutylammonium fluoride in dimethylsulfoxide (DMSO): 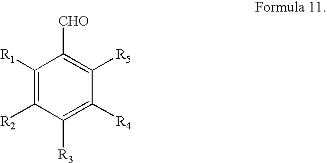
[wherein R₁-R₅ are independently selected from hydrogen, C₁-C₄ alkyl and —N(CH₃)₃⁺⁻OTf (at least one of them is —N(CH₃)₃⁺⁻OTf)]
